(19) United States
(12) Patent Application Publication
(10) Pub. No.: US 2023/0143566 A1
(43) Pub. Date: May 11, 2023

(54) KETO ESTER COMPOSITIONS AND METHODS FOR USING SAME

(71) Applicant: Operade LLC, Jacksonville, FL (US)

(72) Inventors: John N. Catanzaro, Jacksonville, FL (US); Christopher D. Batich, Gainesville, FL (US)

(21) Appl. No.: 17/984,218

(22) Filed: Nov. 9, 2022

Related U.S. Application Data
(60) Provisional application No. 63/277,595, filed on Nov. 9, 2021.

(51) Int. Cl. A61K 31/21; A61K 31/716; A61K 47/55; A61K 31/12

(52) U.S. Cl. CPC A61K 31/21; A61K 31/716; A61K 47/551; A61K 31/12

(57) ABSTRACT

The invention generally relates to keto ester compositions and methods for using the compositions. In one aspect, the invention provides a method for preventing a metabolic-related condition or disorder in a subject, the method comprising the step of providing to the subject an effective amount of at least one keto ester compound or a pharmaceutically acceptable salt thereof, and an effective amount of at least one maltodextrin compound, thereby preventing the metabolic-related condition or disorder. This abstract is intended as a scanning tool for purposes of searching in the particular art and is not intended to be limiting of the present invention.

### RELATED APPLICATIONS

This application claims the benefit of priority to U.S. Provisional Patent Application Ser. No. 63/277,595, filed Nov. 9, 2021, which is hereby incorporated herein by reference in its entirety.

### FIELD OF INVENTION

This disclosure relates to keto ester compositions and methods for using same.

### BACKGROUND

The nutritional state of patients is an important factor in providing successful surgical outcomes. However, patients undergoing surgeries, gastrointestinal procedures, and cardiovascular procedures are often required to maintain NPO (e.g., “nil per os” or nothing by mouth) status for a prolonged period due to risk of peri-operative anesthesia complications such as aspiration. The concept likely dates back to a report from 1946 by Mendelson who described aspiration in obstetric patients not fasting. There is limited data on the optimal duration of NPO status, and the impact of non-adherence to NPO on cancellation of procedures is variable with reports suggesting that anywhere from 2% to 20% of procedures are cancelled or rescheduled due to failure of NPO. There is also limited data on how different components of nutrition affect gastric emptying and safety of conducting procedures after a period of NPO. For example, liquids empty more quickly from the stomach, but are not permitted under NPO. As a result, patient discomfort and dissatisfaction can often develop from feelings of hunger, thirst, and fatigue. Furthermore, they are deprived of nutrition during the pre-operative period, which can prolong post-operative healing times, increase length of hospital stay, and increase risk for infections.

Still further, the metabolic and immune responses to surgical stress when patients are fasting can worsen the catabolic state, cause insulin resistance, cause hyperglycemia, and potentially delay recovery. Insulin resistance, a known risk factor of post-operative complications and increased hospital stay, is a central metabolic change during surgical stress that causes hyperglycemia in patients resulting in stimulation of various endocrine and inflammatory systems. This can result in both an exacerbation of postoperative catabolic state such as loss of body fat and protein stores, and negative effects on various aforementioned measures of patient comfort such as hunger, thirst, fatigue, malaise, pain, anxiety and nausea. Accordingly, patients can require aggressive treatment with insulin to maintain glycemic control and reduce potential organ dysfunction and mortality.

In an effort to ameliorate these harmful effects, certain patients are given a preoperative carbohydrate load. However, large carbohydrate load can potentially cause large spikes in insulin release and related unwanted effects of insulin spikes. Furthermore, medical practitioners may be reluctant to give diabetic patients a large carbohydrate load because of their potential to cause preoperative hyperglycemia and to delay gastric emptying. In certain instances, these undesired effects of carbohydrate loading patients can lead to cancelled surgeries and delayed treatment.

Accordingly, there remains a lack of effective prevention or treatment options for such metabolic-related conditions and disorders. In view of the increasing incidence of such metabolic-related conditions and disorders in surgical patients, the increased cost of health care associated with their treatment and with having to cancel surgeries, there remains a need for effective methods and compositions for the prevention or treatment of metabolic-related conditions and disorders which can produce beneficial effects on the post-operative recovery of patients, measures of patient satiety, and measures of patient comfort or satisfaction, which are also major factors affecting patient adherence to testing. This need and other needs are satisfied by the various aspects of the present disclosure.

### SUMMARY

In accordance with the purpose(s) of the invention, as embodied and broadly described herein, the invention, in one aspect, relates to methods for using keto ester compounds and pharmaceutical and nutraceutical compositions comprising same.

In another aspect, disclosed are methods for preventing or treating various metabolic-related conditions and disorders, for example and without limitation, those caused by or related to surgical stress. The various metabolic-related conditions and disorders can include, without limitation, insulin resistance, hyperglycemia, endocrine system dysfunctions, inflammatory system dysfunctions, dysregulation of glycemic control, gastric emptying, gastric acidity, measures of patient wellbeing and comfort, immunity, clinical outcome and nutrition, and the like.

In another aspect, also disclosed are methods for preventing insulin resistance in a subject, the method comprising the step of providing to the subject an effective amount of at least one keto ester compound or a pharmaceutically acceptable salt thereof, and an effective amount of at least one maltodextrin compound, thereby preventing the subject from insulin resistance.

In another aspect, also disclosed are methods for preventing exacerbation of a postoperative catabolic state in a subject, the method comprising the step of providing to the subject an effective amount of at least one keto ester compound or a pharmaceutically acceptable salt thereof, and an effective amount of at least one maltodextrin compound, thereby preventing the subject from exacerbation of a postoperative catabolic state. In further aspects, the step of providing to may be preoperatively or postoperatively, or a combination thereof.

In another aspect, also disclosed are pharmaceutical and nutraceutical compositions comprising an effective amount of at least one keto ester compound or a pharmaceutically acceptable salt thereof; and an effective amount of at least one maltodextrin compound; and a pharmaceutically acceptable carrier. In at least one aspect, disclosed are preoperative drink compositions comprising an effective amount of at least one keto ester compound or a pharmaceutically acceptable salt thereof; and an effective amount of at least one maltodextrin compound. In various further aspects, the at least one keto ester compound can include salts of beta hydroxy butyric acid.

In another aspect, also disclosed are compositions and methods for improving taste of keto ester compounds and compositions.

Also disclosed are kits comprising the disclosed compounds and compositions. In at least one aspect, disclosed are kits comprising an effective amount of at least one keto ester compound or a pharmaceutically acceptable salt thereof, an effective amount of at least one maltodextrin compound, and one or more of: a) at least one additional agent known to treat a disorder or disease; b) instructions for treating or preventing a metabolic-related condition or disorder; and c) instructions for administering the keto ester compound and the maltodextrin compound in connection with the metabolic-related condition or disorder.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more disorders prior to the administering step. In some aspects of the disclosed methods, the subject has been diagnosed with a need for treatment of one or more metabolic-related conditions or disorders prior to the administering step. In some aspects of the disclosed method, the subject has been diagnosed with requiring a surgical procedure prior to the administering step. In some aspects of the disclosed method, the subject is a preoperative patient prior to the administering step.

As used herein, the term “treatment” or “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e g, a human), and includes: (i) preventing the condition, disorder, or disease from occurring in a subject that can be predisposed to the condition, disorder, or disease but has not yet been diagnosed as having it; (ii) inhibiting the condition, disorder, or disease, i.e., arresting its development; or (iii) relieving the condition, disorder, or disease, i.e., causing regression of it. In one aspect, the subject is a mammal such as a primate, and, in a further aspect, the subject is a human The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. For example, preventing metabolic-related condition or disorder means reducing the incidences, delaying or reversing diseases or disorders that are related to or associated with a metabolic dysfunction.

As used herein, the term “diagnosed” means having been subjected to a physical examination by a person of skill, for example, a physician, and found to have a condition that can be diagnosed or treated by the compounds, compositions, or methods disclosed herein. In some aspects of the disclosed methods, the subject has been diagnosed with a need for treatment of a metabolic-related condition or disorder prior to the administering step. As used herein, the phrase “identified to be in need of treatment for a condition or disorder,” or the like, refers to selection of a subject based upon need for prevention or treatment of the condition or disorder. It is contemplated that the identification can, in one aspect, be performed by a person different from the person making the diagnosis. It is also contemplated, in a further aspect, that the identification can be performed by one who subsequently performed the administration, or a procedure. For example, the administration can also be used for healthy subjects before undergoing a same day procedure or surgery, such as, administration up to 90 minutes prior to colonoscopy or endoscopy.

As used herein, the term “providing” refers to any method of administering or contacting a disclosed compound or composition to a cell, target receptor, or other biological entity. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the terms “metabolic-related disorder” and “metabolic-related condition” refers to disorders, conditions or diseases in which hyperglycemia, hypoglycemia or insulin resistance, anxiety, dehydration, or diarrhea is a major risk factor. In some aspects, metabolic-related conditions or disorders can be based on electrolyte disturbance type, and can include hypokalemia, hypomagnesemia, hyponatremia, hypophosphatemia, hypocalcemia. In other aspects, metabolic-related conditions or disorders can also be classified based on the type of cells involved, and can include: myositis, cellular adenosine triphosphate depletion and hypoxia. In further aspects, metabolic-related conditions or disorders associated with organ and organelle dysfunction include, but are not limited to, pancreatic insufficiency, adrenal insufficiency, hypercortisolemia, hyperinsulinemia, hyperglycemia and the like. In still further aspects, the present invention encompasses, but is not limited to the foregoing conditions, diseases or disorders.

As used herein, “metabolic disorder” refers to a disease, condition or disorder characterized by or exhibiting a metabolic or hormonal dysfunction or imbalance. In further various aspects, the dysfunction may be temporary or may be preexisting. In further various aspects, a metabolic disorder can comprise an immune-mediated disorder, inflammatory disorder, or endocrine disorder, such as, for example, insulin resistance.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half maximal (50%) inhibitory concentration (IC) of a substance.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g., Almarasson, O., et. al. (2004) The Royal Society of Chemistry, 1889-1896. Examples of co-crystals include p-toluenesulfonic acid and benzenesulfonic acid.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

In one aspect, the invention relates to keto ester compounds and compositions for use as agents for the treatment of metabolic-related conditions and disorders in patients undergoing surgery. In a further aspect, the invention relates to compounds and compositions for use in glucose stabilization, increasing alertness, decreasing appetite, and/or increasing satiation. More specifically, in one aspect, the present invention relates to compounds and compositions for the regulation of metabolic and immune response to surgical stress. In another aspect, the present invention also relates to compounds and compositions for the regulation of glycemic control.

It is contemplated that each disclosed derivative can be optionally further substituted. It is also contemplated that any one or more derivative can be optionally omitted from the invention. It is understood that the disclosed compounds can be employed in the disclosed methods of using.

In one aspect, the invention relates to keto ester compounds or a pharmaceutically acceptable salt thereof. In a still further aspect, keto ester compounds include compounds, such as, for example, 3-hydroxybutyl 3-hydroxybutanoate, 3-hydroxybutyl (3R)-3-hydroxybutanoate, 3-hydroxybutyl (R)-3-hydroxybutyrate, R-BHB; BD-AcAc 2; ester ketone, ketone ester; (R)—(R)-3-hydroxybutyl 3-hydroxybutanoate; [(3R)-3-hydroxybutyl] (3R)-3-hydroxybutanoate; (3R)-3-Hydroxybutanoic acid (3R)-3-hydroxybutyl ester; Butanoic acid, 3-hydroxy-, (3R)-3-hydroxybutyl ester, as well as, analogues and salts thereof. In a yet further aspect, the keto ester refers to a compound having the general structure represented by the formula:

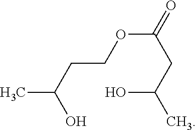

In a further aspect, the keto ester compound is 3-hydroxybutyl (3R)-3-hydroxybutanoate or 3-hydroxybutyl (R)-3-hydroxybutyrate. In a yet further aspect, the keto ester is a compound having the structure represented by the formula:

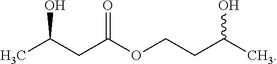

In a further aspect, the keto ester compound can comprise any desired combination of R and S configurations, such as, for example, R,R; R,S; S,R and S,S, and combinations and mixtures thereof. In a yet further aspect, the keto ester compound is the R-acid and the S,R-alcohol (racemic) ester. In a still further aspect, the invention relates to keto esters compounds and analogues thereof, in the form of acids, salts, esters and other derivatives. In an even further aspect, derivatives can include, such as, for example: esters using glycerol as the hydroxyl-group source or other nontoxic molecules. In a further aspect, the least one keto ester compound is 3-hydroxybutyl (R)-3-hydroxybutyrate, or pharmaceutically acceptable forms thereof, and combinations thereof. In a yet further aspect, the at least one keto ester compound can include salts of beta hydroxy butyric acid.

In a further aspect, the invention relates to at least one maltodextrin compound or a pharmaceutically acceptable derivative thereof. In a still further aspects, the maltodextrin compound may comprise a mixture of maltodextrin compounds. In a yet further aspect, maltodextrin refers to polysaccharides comprising D-glucose units connected in chains of variable length. In a still further aspect, the glucose units may be primarily linked with α(1→4) glycosidic bonds. In an even further aspect, the maltodextrin compound may comprise a mixture of chains that vary from 3 to 20 glucose units, for example from 3 to 17 glucose units. In a yet further aspect, at least one maltodextrin compound may comprise a digestion-resistant maltodextrin, delayed-release maltodextrin, or slow-release maltodextrin, or other combinations thereof. In a still further aspect, maltodextrin may comprise a compound having the structure represented by the formula:

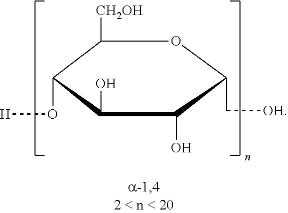

### C. METHODS FOR METABOLIC-RELATED CONDITIONS AND DISORDERS

According to various aspects of the present disclosure, the invention relates to methods for preventing or treating various metabolic-related conditions and disorders, for example and without limitation, those caused by or related to various procedures or surgical stresses. For example, patients undergoing procedures such as surgical, gastroenterological, and cardiovascular procedures often require prolonged fasting due to peri-operative anesthesia risk of complications. Neuro-hormonal changes through this period stimulates hunger and thirst. By overriding this internal response process by maintaining the patient's “NPO status”, it is believed to create psychological discomfort leading to patient dissatisfaction and nutritional deprivation leading to postoperative complications such as infections and delayed wound healing. As detailed herein, typical strategies to address this include carbohydrate loading.

In various further aspects, the present disclosure provides novel treatments and methods that include ketone supplementation in a predictable way which can have a favorable effect on free fatty acids and glucose, without blunting insulin or causing hypoglycemia, and may have a positive effect on mitochondrial function.

For example, in one aspect, the invention relates to methods for preventing or reducing insulin resistance in a subject, the method comprising the step of providing to the subject an effective amount of at least one keto ester compound or a pharmaceutically acceptable salt thereof, and an effective amount of at least one maltodextrin compound, thereby preventing the subject from insulin resistance. In further aspects, the method can further comprise the step of providing an effective amount of at least one electrolyte or vitamin compound or a pharmaceutically acceptable salt thereof. In still further aspects, insulin resistance can comprise a central metabolic change during surgical stress, hyperglycemia, decreased insulin sensitivity, stimulation of various endocrine systems, or stimulation of various inflammatory systems, or combinations thereof.

In another aspect, the invention relates to methods for preventing exacerbation of a postoperative catabolic state in a subject, the method comprising the step of providing to the subject an effective amount of at least one keto ester compound or a pharmaceutically acceptable salt thereof, and an effective amount of at least one maltodextrin compound, thereby preventing the subject from exacerbation of a postoperative catabolic state. In further aspects, the method can further comprise the step of providing an effective amount of at least one electrolyte or vitamin compound or a pharmaceutically acceptable salt thereof. In still further aspects, exacerbation of a postoperative catabolic state can comprise loss of body fat stores, or loss of protein stores, loss of muscle mass, hyperglycemia, or combinations thereof.

In some aspects, activity against metabolic-related conditions and disorders can comprise maintaining a state of calmness; maintaining focus; mitigating acute spikes of hyperglycemia, stimulating, improving, or maintaining insulin release; inhibiting glucagon release, preventing thirst, hunger, mood changes, or a combination thereof.

In a further aspect, the activity against metabolic-related conditions and disorders comprises preventing a metabolic-related condition and disorder. In a still further aspect, the metabolic-related conditions and disorders may be associated with or induced by surgical stress, fasting, or loss of insulin sensitivity. In a still further aspect, the metabolic-related conditions and disorders can comprise an abnormal state of psychology, mental alertness and physiology, or a function-decreasing disorder.

To prevent, treat or control abnormal metabolic-related cellular activity, the compounds and compositions comprising the compounds are provided to a cell, such as a mammalian cell, e.g., a human cell. In a further aspect, providing can comprise administering or contacting the cell with the compounds or compositions. In a still further aspect, prior to providing the compounds or compositions, the cell can be identified with a need for treatment of metabolic-related conditions and disorders, as described herein.

In one aspect, the keto ester compound and the maltodextrin compound are used at various doses. In a further aspect, the keto ester compound dose is from about 0.001 to about 10,000 μM (micromolar) in serum medium. In a still further aspect, the effective amount of the keto ester compound is from about 0.01 to about 1,000 μM in serum medium, including exemplary subranges of about 0.1 to about 100, about 0.1 to about 10 μM, and about 0.2 to about 1 μM. In a yet further aspect, the ratio of keto ester compound to maltodextrin compound is from about 1:1 to about 1:1000, including all subranges therebetween, for example, 1:1, 1:2, 1:3, 1:4, 1:5, 1:6, 1:7, 1:8, 1:9, 1:10, 1:20, 1:25, 1:30, 1:35, 1:50, or 1:100.

In various aspects, and without wishing to be bound by a particular theory, it believed that the specific combination of the keto ester compound and maltodextrin compound, and ratios exhibit the disclosed novel utility for and/or activity against the disclosed metabolic-related conditions and disorders. In a further aspect, the novel activity is not exhibited when the keto ester compound and the maltodextrin compound are used as monotherapy.

In another aspect, the invention also relates to methods for preventing an abnormal physiologic state including electrolyte, carbohydrate or ionic imbalance in a surgical subject, the method comprising the step of preoperatively providing to the subject an effective amount of at least one disclosed composition, thereby preventing the metabolic disorder.

In a yet further aspect, the step of providing to the subject comprises providing to the subject an effective amount of at least one keto ester compound or a pharmaceutically acceptable salt thereof, and an effective amount of at least one maltodextrin compound or a pharmaceutically acceptable salt thereof. In various aspects, and without wishing to be bound by a particular theory, it is believed that the keto ester and maltodextrin compound mitigates an acute carbohydrate glucose surge and flattens the spike caused in the absence of the keto ester compound, which thus exhibit activity against acute hyperglycemia and insulin resistance. In further aspects, the combination of keto ester and maltodextrin compound can increase satiation, decrease thirst, decrease preoperative carbohydrate load, lower insulin release following carbohydrate loading, decrease levels of stress hormones released, improve postoperative surgical outcomes, and the like.

To treat or control the metabolic-related conditions and disorders, the compounds and compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human.

In a further aspect, the subject has been diagnosed with a need for treatment prior to the administering step. In a still further aspect, the subject has been diagnosed with a risk of developing a metabolic-related condition and disorder associated with surgical stress, or fasting, or combinations thereof. Prior to administering the compounds or compositions, the subject can be diagnosed with a need for treatment of metabolic-related conditions and disorders, such as insulin resistance. In a still further aspect, the subject can be identified with a need for treatment of a disclosed treatment, as described herein.

According to various aspects of the disclosure, the disclosed compounds or compositions is generally administered to the subject by oral administration. However, the compounds or compositions can be administered to the subject according to any method. Such methods are well known to those skilled in the art and include, but are not limited to, oral administration, transdermal administration, topical administration, intravaginal administration, and rectal administration. Administration can be continuous or intermittent. A preparation can be administered therapeutically; that is, administered to treat an existing disease or condition. A preparation can also be administered prophylactically; that is, administered for prevention of a disease or condition.

The effective amount or dosage of the compounds can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 1 mg to about 100,000 mg, preferably from about 2 mg to about 20,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days. In some aspects, the compounds can be administered on a preoperative regimen of 1 to 4 times per day, preferably once per day. This dosing regimen can be adjusted to provide the optimal therapeutic response.

In one aspect, the keto ester compound and the maltodextrin compound are used at various doses in a subject. In a further aspect, the keto ester compound and the maltodextrin compound dose is from about 0.1 mg to about 100,000 mg in a subject. In a still further aspect, the effective amount of the keto ester compound is from about 0.1 gm to about 20 gm in a subject, including exemplary subranges of about 0.2 to about 20 gm, about 1 to about 15 gm, about 1 to about 10 gm, about 1 to about 5 gm and about 1 to about 4 gm. In an even further aspect, the effective amount of the maltodextrin compound is from about 1 to about 100 gm in a subject, including exemplary subranges of about 1 to about 50 gm, about 1 to about 25 gm, about 1 to about 20 gm, about 1 to about 15 gm, about 1 to about 10 gm and about 1 to about 5 gm.

In a further aspect, the effective amount of the keto ester compound is from about 0.1 mg to about 100 gm and the effective amount of the maltodextrin compound is from about 1 mg to about 100 gm to a subject. In an even further aspect, the effective amount of the keto ester compound is from about 1 mg to about 50 gm and the effective amount of the maltodextrin compound is from about 1 gm to about 50 gm. In a still further aspect, the effective amount of the keto ester compound is from about 1 to about 25 gm and the effective amount of the maltodextrin compound is from about 1 to about 50 gm. In a yet further aspect, the effective amount of the keto ester compound is from about 1 to about 15 gm and the effective amount of the maltodextrin compound is from about 1 to about 25 gm. In an even further aspect, the ratio of keto ester compound to maltodextrin compound is from about 1:1 to about 1:100.

In determining the effective dose or dosage of the pharmaceutical or nutraceutical composition of the invention, a response to a prophylactic and/or treatment method of the invention can, for example, also be measured by determining the physiological effects of the treatment or medication, such as the decrease or lack of disease symptoms following administration of the treatment or pharmacological agent. Other assays will be known to one of ordinary skill in the art and can be employed for measuring the level of the response. For example, the diagnostic methods that are used to ascertain the likelihood that a subject has or will develop a metabolic-related condition or disorder can be used to ascertain the level of response to a prophylactic and/or treatment method of the invention. The amount of a treatment may be varied for example by increasing or decreasing the amount of a therapeutic composition, by changing the therapeutic composition administered, by changing the route of administration, by changing the dosage timing and so on. The effective amount will vary with the particular condition being treated, the age and physical condition of the subject being treated, the severity of the condition, the duration of the treatment, the nature of the concurrent therapy (if any), the specific route of administration, and the like factors within the knowledge and expertise of the health practitioner. For example, an effective amount can depend upon the degree to which an individual has abnormal levels and/or activity of a physiological process or pathway associated protein or pathway associated protein complex.

The factors involved in determining an effective amount are well known to those of ordinary skill in the art and can be addressed with no more than routine experimentation. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

In some aspects, the effective amount is a therapeutically effective amount. In other aspects, the effective amount is a prophylactically effective amount. In further aspects, the subject is a mammal. In still further aspects, the mammal is a human.

In a further aspect, the method further comprises the step of identifying a subject in need of treatment. In a still further aspect, the subject in need of treatment comprises having at least one risk factor for developing a metabolic-related condition and disorder. In a yet further aspect, the metabolic-related conditions and disorders may be associated with surgical stress, fasting, or anxiety.

In another aspect, the invention also relates to a method for the treatment of a subject, the method comprising the steps of: diagnosing the subject as having a risk factor for a metabolic-related condition and disorder; and administering to the subject an effective amount of at least one keto ester compound or a pharmaceutically acceptable salt thereof, and an effective amount of at least one maltodextrin compound. In some aspects, the risk factor may comprise upcoming exposure to surgical stress, or anxiety, anesthesia, analgesia, or post-operative fatigue, malnutrition, and hunger.

In another aspect, a method of diagnosis comprises performing an experiment upon the subject and identifying a level of a biological marker. In a further aspect diagnosing comprises determining, in a patient, levels of a marker (e.g., concentration of marker; gene expression) indicative of a state of the patient, the state being predictive as to whether the patient will manifest reduced symptoms in response to a treatment.

In a further aspect, the biological marker is a marker for a metabolic-related condition and disorder. In a yet further aspect, the subject is a biological sample. In a still further aspect, the biological sample is selected from a cell, blood, saliva, urine, tissue, or phlegm. In an even further aspects, the biological marker is satiety assessment marker. In a still further aspect, the biological marker comprises GLP-1 level, Ghrelin level, and leptin level. insulin level, or blood glucose level, or a combination thereof.

In one aspect, diagnosis of a metabolic-related condition and disorder comprises a medical history. In a further aspect, the diagnosis comprises comparing the findings of the medical history with the diagnostic standards. In a still further aspect, the diagnosis comprises finding of at least one risk factor for developing a metabolic-related condition and disorder. In a yet further aspect, the metabolic-related condition or disorder is associated with surgical stress, fasting, or endocrine dysfunction. In an even further aspect, the risk factor comprises surgical stress, or anxiety, hypoxia, insulin resistance.

In further aspects, the disclosed methods are effective to treat or improve satiety in the subject. In still further aspects, satiety can be assessed by measuring GLP-1 levels, ghrelin levels, and leptin levels, insulin and blood glucose levels, and combinations thereof. Without wishing to be bound by a particular theory, insulin and blood glucose levels can also give an idea of nutritional intake over time, with the caveat that glucose homeostasis mechanisms generally maintain euglycemia in healthy patients.

### D. COMPOSITIONS

In one aspect, the invention relates to compositions comprising an effective amount of at least one keto ester compound or a pharmaceutically acceptable analogue thereof; and an effective amount of at least one maltodextrin compound. In further aspects, the compositions can comprise an effective amount of at least one electrolyte or vitamin compound or a pharmaceutically acceptable salt thereof. In some aspects, the composition can comprise pharmaceutical compositions. In other aspects, the composition can comprise nutraceutical compositions.

The compounds generally have IC₅₀ values ranging from about 0.01 micromolar to about 100 millimolar. IC₅₀ refers to the concentration of the compound that is required for 50% antagonism or inhibition of the target in vitro. IC₅₀ also refers to the concentration of a substance that is required for 50% antagonism or inhibition of the target in vivo. The activity of the compounds, including IC₅₀ may be determined according to procedures known in the art.

The pharmaceutical and nutraceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In various aspects, the disclosed pharmaceutical and nutraceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The compositions can be conveniently presented in unit dosage form and prepared by any of the methods well known in the art of pharmacy.

Pharmaceutical compositions of the present invention suitable for enteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

In various aspects, the pharmaceutical and nutraceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical and nutraceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of preoperative and postoperative administration, suspensions, elixirs and solutions are the preferred oral form and tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In further aspects, the formulations described above can include, as appropriate, one or more additional nutritional ingredients such as protein, amino acids, vitamins, minerals, anti-oxidants, trace elements, and the like. For example, in further aspects, the compositions may comprise whey protein in a range from about 1.0 to 12.0 wt. %, such as 3.0-11.0 wt. %, 4.0-10.0 wt. %, 4.0-10.0 wt. %, 5.0-9.0 wt. %, and 4.5-8.5 wt. %. As used herein, whey protein is understood to comprise any protein present or derived from whey, including beta-lactoglobulin, alpha-lactoalbumin, serum albumin and immunoglobulins and any combination of these in any proportion. The whey and whey proteins may be derived from any mammal, including ruminants, in particular, cattle, buffalo, horse, sheep and goat, preferably cattle (bovine whey). The whey protein may comprise a whey protein concentrate, (WPC), whey protein isolate (WPI, having 90% or more proteins in total solids). Whey protein, WPI or WPC can be derived from any type of whey, for example, cheese whey, acid whey, whey whey, cottage cheese whey. In a further aspect, the whey protein may comprise a WPI or WPC derivative from acid casein production. The whey protein may be an integral part of another protein, such as a milk protein isolate or a milk protein concentrate. In a still further aspect, the amino acid may comprise arginine, histidine, leucine, valine, cysteine or glutamine., and combinations thereof.

In a yet further aspect, the minerals, vitamins, antioxidants, and trace elements are present at a level between 0.1 times and 10 times, such as between 0.25 and 5 times or 0.5 and 2.5 times of the recommended daily intake per liter, serving, dose, or bottle. In an even further aspect, the minerals, vitamins, antioxidants, and trace elements may comprise one or more of: Vitamin A, Vitamin B-1, Vitamin B-2, Vitamin B-6, Vitamin B-12, Vitamin C, Vitamin D, Vitamin E, Niacinamide, Para-aminobenzoic acid (PABA), Pantothenic Acid, Choline Bitartrate, Inositol, Rutin, Betaine Hydrochloride, Folic Acid, Biotin, Calcium, Iron, Magnesium, Zinc, Potassium, Manganese, Iodine, Chromium, Copper, and Selenium.

In addition to the aforementioned nutritional ingredients, the formulations described above can include, as appropriate, one or more additional carrier ingredients such as diluents, buffers, flavoring agents, binders, surface-active agents, thickeners, lubricants, preservatives (including anti-oxidants) and the like. For example, the compositions may comprise from greater than 0.01 wt. % to about 1 wt. % of a flavoring, such as natural grape (white) flavor, natural strawberry flavor, natural watermelon flavor, natural raspberry (blue) flavor, natural bubble gum flavor, or the like. In some embodiments, the composition is clear or colorless, or otherwise compatible for use in GI procedures.

Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, the effective amount is a therapeutically effective amount. In a still further aspect, the effective amount is a prophylactically effective amount. In a yet further aspect, the composition is formulated for oral administration. In an even further aspect, the composition is formulated for enteral administration. In a still further aspect, the keto ester compound may be present in an amount from about 0.1 wt. % keto ester to about 40 wt. %, including any % or subrange % therebetween, for example from about 0.15, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 3.0, 3.1, 3.2 3.3, 3.4, 3.5, 3.6, 3.7, 3.8, 3.9, 4.0, 4.1, 4.2 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 wt. %. In a yet further aspect, the maltodextrin compound may be present in an amount from about 0.1 wt. % to about 40 wt. %, including any % or subrange % therebetween, for example from about 0.15, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 3.0, 3.1, 3.2 3.3, 3.4, 3.5, 3.6, 3.7, 3.8, 3.9, 4.0, 4.1, 4.2 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 wt. %.

Keto ester compounds are known to be bitter-tasting and to cause gastrointestinal (GI) distress after ingestion. Additionally, the bitterness of ketone ester compounds can cause complete patient intolerance and noncompliance, particularly with larger doses, such as those in described in the disclosed methods.

In various further aspects, the present disclosure also provides compositions and methods for reducing bitterness and/or improving taste of keto ester compounds and compositions. In still further aspects of the invention, the keto ester compound and maltodextrin compound are co-formulated prior to preparation of a final composition. It has surprising been found that co-formulating the keto ester compound and maltodextrin compound together to produce a first composition substantially reduces the bitterness of the keto ester compound when said first composition is introduced in a final composition. For example, in a further aspect, the keto ester compound is formulated with the maltodextrin compound to produce a 21% keto ester in maltodextrin composition. In a still further aspect, the keto ester compound may be formulated with the maltodextrin compound to produce a first co-formulation comprising from about 1% keto ester to about 40% keto ester in maltodextrin composition, including any % or subrange % therebetween. In some aspects, the keto ester compound may be formulated with the maltodextrin compound to produce a first co-formulation comprising a powder. In other aspects, the keto ester compound may be formulated with the maltodextrin compound to produce a first co-formulation comprising a powder.

Without wishing to be bound by a particular theory, it is believed the compositions comprising the co-formulated keto ester compound and maltodextrin compound exhibit substantially reduced bitterness, improved patient tolerance, and reduced gastrointestinal (GI) distress after ingestion, particularly when compared to substantially identical formulation without a co-formulated keto ester and maltodextrin composition. In various aspects, the at least one maltodextrin compound may comprise a digestion-resistant maltodextrin, delayed-release maltodextrin, or slow-release maltodextrin, or other combinations thereof.

In one embodiment, an example formulation of a composition in accordance with present invention can comprise:

| | | Percent by |
|---|---|---|
| Material ID | Ingredients for a 12 oz (c. 340 g) serving | weight |
| 210427-52 | ketone ester (on 21% maltodextrin) | 2.2540% |
| 1755813 | maltodextrin | 5.6000% |
| 1756373 | monopotassium phosphate-45 mg per 12 oz serving | 0.0823% |
| 2418014 | sodium citrate-188 mg per 12 oz serving | 0.2258% |
| 1752373 | citric acid | 0.0900% |
| 837001 | malic acid | 0.0900% |
| 401994 | Prinova vitamin blend-(50% Vitamin A, 10% Selenium, | 0.0085% |
| | 35% Zinc per 12 oz serving) | |
| 1958045 | stevia extract (180 ppm) | 0.0180% |
| BQ-393-336-2 | Flavor-Natural Juice Booster flavor | 0.0500% |
| ZM-299-151-2 | Flavor-Natural Sweetness Modifier flavor | 0.0500% |
| | Water | 91.5314% |

In further aspects, an example formulation can be prepared by adding 5-15 mls of a keto ester-maltodextrin co-formulation above for a 12 or 16 oz drink in a bottle made of PFA (perfluoroalkoxy alkane), which will allow a hot-fill pasteurization. For the calculation of 5 mls of keto ester co-formulated composition in 16 oz bottle, there will be about 66.01 mmolar of the keto ester, and for the 15 mls in 12 oz bottle there will be about 264 mmolar of the keto ester compound.

The preparation process is Hot Fill, 190-195 F. Flavors include: Natural Grape (white) flavor @ 0.12%; Natural Strawberry flavor, @ 0.12%; Natural Watermelon Flavor, @ 0.12%; Natural Raspberry (blue) flavor, @ 0.13%; Natural Bubble Gum flavor, @ 0.12%.

In another aspect, a double concentrated example formulation of a composition in accordance with present invention can comprise:

| | | Percent by |
|---|---|---|
| Material ID | Ingredients | weight |
| 210427-52 | ketone ester (on 21% maltodextrin) | 4.5080% |
| 1755813 | maltodextrin | 5.6000% |
| 1756373 | monopotassium phosphate-45 mg per 12 oz serving | 0.0823% |
| 2418014 | sodium citrate-188 mg per 12 oz serving | 0.2258% |
| 1752373 | citric acid | 0.0900% |
| 837001 | malic acid | 0.0900% |
| 401994 | Prinova ™ vitamin blend-(50% Vitamin A, 10% Selenium, | 0.0085% |
| | 35% Zinc per 12 oz serving) | |
| 1958045 | stevia extract (180 ppm) | 0.0180% |
| BQ-393-336-2 | Flavor-Natural Juice Booster flavor | 0.0500% |
| ZM-299-151-2 | Flavor-Natural Sweetness Modifier flavor | 0.0500% |
| | water | 89.2774% |

In another aspect, an example formulation of a composition in accordance with present invention can comprise:

| | | Percent by |
|---|---|---|
| Material ID | Ingredients | weight |
| 210427-52 | Natural Advantage ketone ester (on 21% maltodextrin) | 2.2540% |
| 1755813 | maltodextrin-copacker/supplier | 5.6000% |
| 1756373 | monopotassium phosphate-copacker/supplier | 0.0823% |
| | 45 mg per 12 oz serving | |
| 2418014 | sodium citrate - copacker/supplier | 0.0553% |
| | 188 mg per 12 oz serving | |
| 1752373 | citric acid-copacker | 0.118% |
| 837001 | malic acid-copacker | 0.118% |
| 401994 | Prinova vitamin blend-(50% Vitamin A, 10% Selenium, 35% | 0.0085% |
| | Zinc per 12 oz serving) | |
| 1958045 | stevia extract (180 ppm)-copacker | 0.0180% |
| BQ-393-336-2 | Givaudan Flavor-Natural Juice Booster flavor | 0.0500% |
| ZM-299-151-2 | Givaudan Flavor-Natural Sweetness Modifier flavor | 0.0500% |
| | water | 91.4804% |

The process is Hot Fill, 190-195 F for 30 second hold, acidified (<4.2 pH), 12 oz (355 ml) bottle with an assumed weight between 364-366 grams.

It is understood that the disclosed compositions can be prepared from the disclosed compounds. It is also understood that the disclosed compositions can be employed in the disclosed methods of using.

### E. KITS

In one aspect, the invention relates to a kit comprising an effective amount of at least one keto ester compound or a pharmaceutically acceptable salt thereof, an effective amount of at least one maltodextrin compound, and one or more of: a) instructions for treating or preventing a metabolic-related condition or disorder; and b) instructions for administering the keto ester compound and the maltodextrin compound in connection with the metabolic-related condition or disorder. In further aspects, the administering is preoperative administration. In still further aspects, the administering is postoperative administration. In yet further aspects, the administering is preoperative and postoperative administration.

The kits can also comprise compounds co-packaged, co-formulated, and/or co-delivered with other components. For example, a drug manufacturer, a drug reseller, a physician, a compounding shop, or a pharmacist can provide a kit comprising a disclosed compound and another component for delivery to a patient.

In a further aspect, the metabolic-related condition or disorder is associated with surgical stress and/or fasting. In a still further aspect, the metabolic-related condition or disorder comprises predisposition to a disclosed metabolic-related condition or disorder. In a yet further aspect, the metabolic-related condition or disorder is selected from medical history or family history.

In a further aspect, the compounds and the at least one additional agent are co-packaged. In a still further aspect, the compounds and the at least one additional agent are co-formulated. In a yet further aspect, each dose of the keto ester compound and the maltodextrin compound are co-formulated. In an even further aspect, each dose of the keto ester compound, the maltodextrin compound, and the at least one agent are co-formulated. In a still further aspect, each dose of the keto ester compound and the at least one agent are co-formulated. In a yet further aspect, each dose of the keto ester-maltodextrin co-formulation and the at least one additional agent are co-packaged.

In a further aspect, the kit may comprise a plurality of dosage forms, the plurality comprising one or more doses; wherein each dose comprises an effective amount of the keto ester compound and the maltodextrin compound. In a still further aspect, each dose comprises an effective amount of the keto ester compound, the maltodextrin compound, and at least one additional agent.

In a further aspect, each dose of the keto ester compound and the maltodextrin compound are administered sequentially. In a still further aspect, each dose of the keto ester compound and the maltodextrin compound are administered simultaneously. In a yet further aspect, each dose of the keto ester compound and the at least one additional agent are administered sequentially. In an even further aspect, each dose of the keto ester-maltodextrin co-formulation and the at least one additional agent are administered simultaneously. In a still further aspect, each dose of the keto ester-maltodextrin composition, and the at least one agent are administered sequentially. In a yet further aspect, each dose of the keto ester-maltodextrin composition, and the at least one agent are administered simultaneously.

In a still further aspect, the effective amount is a therapeutically effective amount. In yet a further aspect, the effective amount is a prophylactically effective amount.

In a further aspect, the dosage forms are formulated for oral administration, and/or enteral administration. In a still further aspect, the dosage form for the compounds is formulated for oral administration. In yet a further aspect, the dosage form for the compound is formulated for oral administration and the dosage form for the at least one agent is formulated for oral administration. In an even further aspect, the dosage form for the compounds is formulated for oral administration as a drink.

In a further aspect, the additional agent can comprise nutritional supplements, anti-inflammatory medicines (e.g., NSAIDS) antiplatelet medicines (e.g., clopidogrel, aspirin), anticoagulants (e.g., warfarin, heparin), lipid lowering medicines (e.g., statins, niacin), anti-hypertensive medicines (e.g., angiotensin-converting enzyme (ACE) inhibitors, angiotensin II receptor blockers (ARBs), beta-blockers, diuretics, and calcium channel blockers (CCBs)), anti-diabetic medicines (e.g., metformin), chemotherapeutic agents, or a combination thereof. In a still further aspect, the nutritional supplement can comprise vitamins, minerals, antioxidants, amino acids, fatty acid complex, digestive enzymes, or a combination thereof.

It is understood that the disclosed kits can be prepared from the disclosed compounds, products, and pharmaceutical compositions. It is also understood that the disclosed kits can be employed in connection with the disclosed methods of using.

The disclosed compositions and methods include at least the following aspects:

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way.

### 1. Example 1: Pre-Operative Treatment Study

In this prophetic example, the effect of treatment with a disclosed composition comprising a keto ester compound and maltodextrin may be evaluated on patients.

The general study methods and aims are as follows:

Aim 1—To determine whether objective measures of satiety can be improved by administration of a disclosed pre-operative nutritional composition solution (“OperAde”) comprising a keto ester compound and maltodextrin in healthy individuals, without adversely affecting gastric emptying.

Aim 2—To assess patient satiety by administering an in-house novel questionnaire before and after ingestion of the OperAde solution.

In this Example, randomized healthy volunteers are to either receive OperAde comprising a keto ester compound and maltodextrin or placebo solution.

Study Measures:

1) Ketones (acetoacetate, B-hydroxybutyrate, and acetone), GIP, GLP-1, CCK, leptin, insulin, glucose, and ghrelin levels 30 mins before and 60, 120, 240, 480 mins after consuming OperAde, as well as acetaminophen levels 60, 120, 240, 480 minutes after ingestion.
2) Glycemic index and glycemic load of OperAde
3) Patient satiety

All patients will be registered for this study and undergo the necessary testing. The study is powered to detect significant changes in satiety hormones as outlined above. Based on an expected change in hormone levels of 50% from baseline in the OperAde group compared to 20% from baseline in the Placebo group and setting alpha 0.05 and beta at 0.8. Total sample size is 72 subjects, 36 in each group. Group 1 will get Placebo solution—36 subjects; Group 2 will get OperAde solution—36 subjects.

Inclusion and Exclusion Criteria:

Inclusion criteria: age>18, normal BMI (18 to 30), and volunteers without any known medical problems. Exclusion criteria include age<18 years old, pregnancy, BMI>30, and underlying medical problems such as DM, gastrointestinal motility issues, history of abdominal surgeries, narcotic medications within 2 weeks, illicit drug use within 2 weeks.

Study Timelines:

The expected duration of data collection and analysis will last approximately 3 months.
a) Study procedures and data analysis
Subjects will present to a single study related visit. Due to circadian rhythm variation in ghrelin and leptin levels in correlation to hunger, patients should be NPO for a period of at least 8 hours starting at midnight and have blood drawn at 8 am. Baseline bloods (as above) are to be drawn 30 minutes prior to ingestion of the study product. A brief questionnaire is to be completed. OperAde (or placebo) will be consumed by the subject under observation. A repeat blood draw and questionnaire will be administered 60, 120, 240, and 480 minutes after consumption of the product. Data will be analyzed using student t-test and chi-squared test.

Blood collection: Blood collection will consist of 2×10 mL (EDTA) tubes. Data will be collected at baseline and during the time points mentioned above (30 minutes prior to consumption and 60, 120, 240, 480 minutes after consumption).

Data Recorded Will Include:

### Age

### Gender

### Height

### Weight

### BMI

### Race/Ethnicity

### Self-reported Height

### Self-reported Weight

History of esophageal/gastric surgical procedure(s)
History of diabetes
History of GI or Endocrine related disorder(s)
Medications currently taking

Treatment Benefits:

The effect of OperAde on healthy individuals without the effect of the stress of procedures will be assessed. Next phase is to study its effects on patients undergoing surgeries, gastrointestinal procedures, and cardiovascular procedures. Without wishing to be bound by a particular theory, the study is expected to show OperAde improves satiety and surgical outcomes, such as improved dysregulation of glycemic control, gastric emptying, gastric acidity, measures of patient wellbeing and comfort, immunity, clinical outcome and/or nutrition.

The present invention includes at least the following aspects: Aspect 1: A method preventing or treating a metabolic-related condition or disorder in a subject, the method comprising the step of providing to the subject an effective amount of at least one keto ester compound or a pharmaceutically acceptable salt thereof, and an effective amount of at least one maltodextrin compound, thereby preventing or treating the metabolic-related condition or disorder.

Aspect 2: A method for reducing or preventing insulin resistance in a subject, the method comprising the step of providing to the subject an effective amount of at least one keto ester compound or a pharmaceutically acceptable salt thereof, and an effective amount of at least one maltodextrin compound, thereby reducing or preventing the subject from insulin resistance.

Aspect 3: A method for treating or preventing exacerbation of a postoperative catabolic state in a subject, the method comprising the step of providing to the subject an effective amount of at least one keto ester compound or a pharmaceutically acceptable salt thereof, and an effective amount of at least one maltodextrin compound, thereby treating or preventing the subject from exacerbation of a postoperative catabolic state.

Aspect 4: A method for preventing or treating a metabolic-related condition or disorder in a postoperative subject, the method comprising the step of providing to the subject after a surgical procedure an effective amount of at least one keto ester compound or a pharmaceutically acceptable salt thereof, and an effective amount of at least one maltodextrin compound, thereby treating or preventing the a metabolic-related condition or disorder.

Aspect 5: The method of any preceding aspect, wherein the method further comprises the step of providing an effective amount of at least one electrolyte or vitamin compound or a pharmaceutically acceptable salt thereof.

Aspect 6: The method of any preceding aspect, wherein the metabolic-related condition or disorder comprises insulin resistance, hyperglycemia, endocrine system dysfunctions, inflammatory system dysfunctions, dysregulation of glycemic control, gastric emptying, gastric acidity, measures of patient wellbeing and comfort, immunity, clinical outcome and nutrition.

Aspect 7: The method of any preceding aspect, wherein insulin resistance can comprise a central metabolic change during surgical stress, hyperglycemia, decreased insulin sensitivity, stimulation of various endocrine systems, or stimulation of various inflammatory systems, or combinations thereof.

Aspect 8: The method of any preceding aspect, wherein exacerbation of a postoperative catabolic state can comprise loss of body fat stores, loss of protein stores, loss of muscle mass, or hyperglycemia, or combinations thereof.

Aspect 9: The method of any preceding aspect, wherein the at least one keto ester compound is selected from 3-hydroxybutyl 3-hydroxybutanoate, 3-hydroxybutyl (3R)-3-hydroxybutanoate, 3-hydroxybutyl (R)-3-hydroxybutyrate, R-BHB; BD-AcAc 2; ester ketone, ketone ester; (R)—(R)-3-hydroxybutyl 3-hydroxybutanoate; [(3R)-3-hydroxybutyl] (3R)-3-hydroxybutanoate; (3R)-3-Hydroxybutanoic acid (3R)-3-hydroxybutyl ester; Butanoic acid, 3-hydroxy-, (3R)-3-hydroxybutyl ester, analogues and salts thereof.

Aspect 10: The method of any preceding aspect, wherein the at least one keto ester compound is a compound having the general structure represented by the formula:

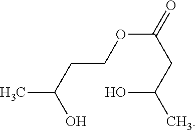

Aspect 11: The method of any preceding aspect, wherein the at least one keto ester compound is 3-hydroxybutyl (3R)-3-hydroxybutanoate or 3-hydroxybutyl (R)-3-hydroxybutyrate.

Aspect 12: The method of any preceding aspect, wherein the at least one keto ester compound is a compound having the structure represented by the formula:

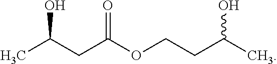

Aspect 13: The method of any preceding aspect, wherein the at least one keto ester compound is a R-acid and S,R-alcohol (racemic) ester.

Aspect 14: The method of any preceding aspect, wherein the at least one maltodextrin compound is a polysaccharide comprising D-glucose units connected in chains of variable length.

Aspect 15: The method of any preceding aspect, wherein the at least one maltodextrin compound comprises glucose units primarily linked with α(1→4) glycosidic bonds.

Aspect 16: The method of any preceding aspect, wherein the at least one maltodextrin compound comprises a mixture of chains that vary from 3 to 17 glucose units.

Aspect 17: The method of any preceding aspect, wherein the at least one maltodextrin compound comprises a compound having the structure represented by the formula:

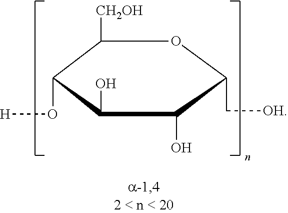

Aspect 18: The method of any preceding aspect, wherein the at least one maltodextrin compound comprises a digestion-resistant maltodextrin, delayed-release maltodextrin, or slow-release maltodextrin, or other combinations thereof.

Aspect 19: The method of any preceding aspect, wherein the metabolic-related condition or disorder is associated with surgical stress or fasting, or a combination thereof.

Aspect 20: The method of any preceding aspect, further comprising the step of identifying a cell or subject in need of treatment or prevention.

Aspect 21: The method of any preceding aspect, wherein the effective amount of the at least one keto ester compound is from about 0.001 gm to about 100 gm.

Aspect 22: The method of any preceding aspect, wherein the effective amount of the at least one maltodextrin compound is from about 0.01 gm to about 100 gm.

Aspect 23: The method of any preceding aspect, wherein the effective amount of the keto ester compound are from about 0.001 to about 10000 μM in serum medium.

Aspect 24: The method of any preceding aspect, wherein the effective amount of the keto ester compound is from about 0.01 to about 1000 μM in serum medium.

Aspect 25: The method of any preceding aspect, wherein the effective amount of the keto ester compound is from about 0.1 to about 100 μM in serum medium.

Aspect 26: The method of any preceding aspect, wherein the ratio of keto ester compound to maltodextrin compound is from about 1:1 to about 1:1000.

Aspect 27: The method of any preceding aspect, wherein the ratio of keto ester compound to maltodextrin compound is from about 1:1 to about 1:25.

Aspect 28: The method of any preceding aspect, wherein the ratio of keto ester compound to maltodextrin compound is from about 1:1 to about 1:8.

Aspect 29: The method of any preceding aspect, wherein the ratio of keto ester compound to maltodextrin compound is from about 1:1 to about 1:4.

Aspect 30: The method of any preceding aspect, wherein the ratio of keto ester compound to maltodextrin compound is from about 1:1 to about 1:2.

Aspect 31: The method of any preceding aspect, wherein the at least one keto ester compound and the at least one maltodextrin compound are co-formulated.

Aspect 32: The method of any preceding aspect, wherein the at least one keto ester compound and the at least one maltodextrin compound are co-formulated as a powder.

Aspect 33: The method of any preceding aspect, wherein the at least one keto ester compound and the at least one maltodextrin compound are co-formulated in solution.

Aspect 34: The method of any preceding aspect, wherein the co-formulated ratio of keto ester compound to maltodextrin compound is from about 1:1 to about 1:25.

Aspect 35: The method of any preceding aspect, wherein the co-formulated ratio of keto ester compound to maltodextrin compound is from about 1:1 to about 1:8.

Aspect 36: The method of any preceding aspect, wherein the co-formulated ratio of keto ester compound to maltodextrin compound is from about 1:1 to about 1:4.

Aspect 37: The method of any preceding aspect, wherein the co-formulated ratio of keto ester compound to maltodextrin compound is from about 1:1 to about 1:2.

Aspect 38: The method of any preceding aspect, wherein the effective amount is a therapeutically effective amount.

Aspect 39: The method of any preceding aspect, wherein the effective amount is a prophylactically effective amount.

Aspect 40: The method of any preceding aspect, wherein the subject is a mammal.

Aspect 41: The method of any preceding aspect, wherein the mammal is a human.

Aspect 42: The method of any preceding aspect, further comprising the step of identifying a subject in need of prevention or treatment of a metabolic-related condition or disorder.

Aspect 43: The method of any preceding aspect, wherein the subject in need of treatment comprises having at least one risk factor for developing a metabolic-related condition or disorder.

Aspect 44: The method of any preceding aspect, wherein the at least one risk factor is an upcoming surgical procedure.

Aspect 45: The method of any preceding aspect, wherein the step of providing comprises providing at least one hour prior to a surgical procedure.

Aspect 46: The method of any preceding aspect, wherein the step of providing comprises providing from at least about one minute to 24 hours prior to a surgical procedure.

Aspect 47: The method of any preceding aspect, wherein the step of providing comprises providing after a surgical procedure.

Aspect 48: The method of any preceding aspect, wherein the step of providing comprises providing from at least about one minute to 24 hours after to a surgical procedure.

Aspect 49: A composition comprising an effective amount of at least one keto ester compound or a pharmaceutically acceptable salt thereof, and an effective amount of at least one maltodextrin compound.

Aspect 50: The composition of any preceding aspect, wherein the at least one keto ester compound is selected from 3-hydroxybutyl 3-hydroxybutanoate, 3-hydroxybutyl (3R)-3-hydroxybutanoate, 3-hydroxybutyl (R)-3-hydroxybutyrate, R-BHB; BD-AcAc 2; ester ketone, ketone ester; (R)—(R)-3-hydroxybutyl 3-hydroxybutanoate; [(3R)-3-hydroxybutyl] (3R)-3-hydroxybutanoate; (3R)-3-Hydroxybutanoic acid (3R)-3-hydroxybutyl ester; Butanoic acid, 3-hydroxy-, (3R)-3-hydroxybutyl ester, analogues and salts thereof.

Aspect 51: The composition of any preceding aspect, wherein the at least one keto ester compound is a compound having the general structure represented by the formula:

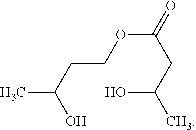

Aspect 52: The composition of any preceding aspect, wherein the at least one keto ester compound is 3-hydroxybutyl (3R)-3-hydroxybutanoate or 3-hydroxybutyl (R)-3-hydroxybutyrate.

Aspect 53: The composition of any preceding aspect, wherein the at least one keto ester compound is a compound having the structure represented by the formula:

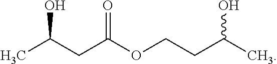

Aspect 54: The composition of any preceding aspect, wherein the at least one keto ester compound is a R-acid and the S,R-alcohol (racemic) ester.

Aspect 55: The composition of any preceding aspect, wherein the at least one maltodextrin compound is a polysaccharide comprising D-glucose units connected in chains of variable length.

Aspect 56: The composition of any preceding aspect, wherein the at least one maltodextrin compound comprises glucose units primarily linked with α(1→4) glycosidic bonds.

Aspect 57: The composition of any preceding aspect, wherein the at least one maltodextrin compound comprises a mixture of chains that vary from 3 to 17 glucose units.

Aspect 58: The composition of any preceding aspect, wherein the at least one maltodextrin compound comprises a compound having the structure represented by the formula:

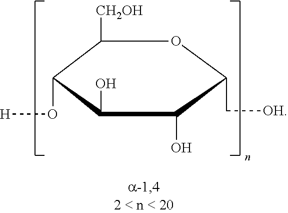

Aspect 59: The composition of any preceding aspect, wherein the at least one maltodextrin compound comprises a digestion-resistant maltodextrin, delayed-release maltodextrin, or slow-release maltodextrin, or other combinations thereof.

Aspect 60: The composition of any preceding aspect, wherein the effective amount of the at least one keto ester compound is from about 0.01 gm to about 100 gm.

Aspect 61: The composition of any preceding aspect, wherein the effective amount of the at least one maltodextrin compound is from about 0.01 gm to about 100 gm.

Aspect 62: The composition of any preceding aspect, wherein the ratio of keto ester compound to maltodextrin compound is from about 1:1 to about 1:1000.

Aspect 63: The composition of any preceding aspect, wherein the ratio of keto ester compound to maltodextrin compound is from about 1:1 to about 1:25.

Aspect 64: The composition of any preceding aspect, wherein the at least one keto ester compound and the at least one maltodextrin compound are co-formulated.

Aspect 65: The method of any preceding aspect, wherein the at least one keto ester compound and the at least one maltodextrin compound are co-formulated as a powder.

Aspect 66: The method of any preceding aspect, wherein the at least one keto ester compound and the at least one maltodextrin compound are co-formulated in solution.

Aspect 67: The composition of any preceding aspect, wherein the co-formulated ratio of keto ester compound to maltodextrin compound is from about 1:1 to about 1:25.

Aspect 68: The composition of any preceding aspect, wherein the co-formulated ratio of keto ester compound to maltodextrin compound is from about 1:1 to about 1:8.

Aspect 69: The composition of any preceding aspect, wherein the co-formulated ratio of keto ester compound to maltodextrin compound is from about 1:1 to about 1:4.

Aspect 70: The composition of any preceding aspect, wherein the co-formulated ratio of keto ester compound to maltodextrin compound is from about 1:1 to about 1:2.

Aspect 71: The composition of any preceding aspect, wherein the effective amount is a therapeutically effective amount.

Aspect 72: The composition of any preceding aspect, wherein the effective amount is a prophylactically effective amount.

Aspect 73: The composition of any preceding aspect, wherein the effective amount is an amount effective to prevent or treat a metabolic-related condition or disorder.

Aspect 74: The composition of any preceding aspect, further comprising at least one of: monopotassium phosphate, sodium citrate, citric acid, malic acid, a vitamin blend such as (50% Vitamin A, 10% Selenium, 35% Zinc per serving), a sweetener such as stevia extract, a flavoring, and water.

Aspect 75: The composition of any preceding aspect, wherein the composition is a pharmaceutical composition.

Aspect 76: The composition of any preceding aspect, wherein the composition is a nutraceutical composition.

Aspect 77: The composition of any preceding aspect, wherein the composition is formulated as a preoperative drink.

Aspect 78: The method or composition of any preceding aspect, wherein the composition is a formulation disclosed in the specification.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications may be referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein can be different from the actual publication dates, which can require independent confirmation.

The patentable scope of the invention is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims. It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for treating or preventing exacerbation of a postoperative catabolic state in a subject, the method comprising the step of providing to the subject an effective amount of at least one keto ester compound or a pharmaceutically acceptable salt thereof, and an effective amount of at least one maltodextrin compound, thereby treating or preventing the subject from exacerbation of a postoperative catabolic state.

2. The method of claim 1, wherein the method further comprises the step of providing an effective amount of at least one electrolyte or vitamin compound or a pharmaceutically acceptable salt thereof.

3. The method of claim 2, wherein exacerbation of a postoperative catabolic state comprises loss of body fat stores, loss of protein stores, loss of muscle mass, or hyperglycemia, or combinations thereof.

4. The method of claim 3, wherein the at least one keto ester compound is selected from 3-hydroxybutyl 3-hydroxybutanoate, 3-hydroxybutyl (3R)-3-hydroxybutanoate, 3-hydroxybutyl (R)-3-hydroxybutyrate, R-BHB; BD-AcAc 2; ester ketone, ketone ester; (R)—(R)-3-hydroxybutyl 3-hydroxybutanoate; [(3R)-3-hydroxybutyl] (3R)-3-hydroxybutanoate; (3R)-3-Hydroxybutanoic acid (3R)-3-hydroxybutyl ester; Butanoic acid, 3-hydroxy-, (3R)-3-hydroxybutyl ester, analogues and salts thereof.

5. The method of claim 4, wherein the at least one maltodextrin compound comprises a digestion-resistant maltodextrin, delayed-release maltodextrin, or slow-release maltodextrin, or combinations thereof.

6. The method of claim 5, wherein the at least one keto ester compound and the at least one maltodextrin compound are co-formulated; and wherein the co-formulated ratio of keto ester compound to maltodextrin compound is from about 1:1 to about 1:25.

7. The method of claim 6, wherein the at least one keto ester compound is 3-hydroxybutyl (3R)-3-hydroxybutanoate or 3-hydroxybutyl (R)-3-hydroxybutyrate.

8. The method of claim 7, wherein the step of providing comprises providing from at least about one minute to 24 hours prior to a surgical procedure.

9. A method for preventing or treating a metabolic-related condition or disorder in a postoperative subject, the method comprising the step of providing to the subject after a surgical procedure an effective amount of at least one keto ester compound or a pharmaceutically acceptable salt thereof, and an effective amount of at least one maltodextrin compound, thereby treating or preventing the a metabolic-related condition or disorder; wherein the metabolic-related condition or disorder is associated with surgical stress or fasting, or a combination thereof.

10. The method of claim 9, wherein the method further comprises the step of providing an effective amount of at least one electrolyte or vitamin compound or a pharmaceutically acceptable salt thereof.

11. The method of claim 10, wherein the metabolic-related condition or disorder comprises insulin resistance, hyperglycemia, endocrine system dysfunctions, inflammatory system dysfunctions, dysregulation of glycemic control, gastric emptying, gastric acidity, measures of patient wellbeing and comfort, immunity, clinical outcome and nutrition, and combinations thereof.

12. The method of claim 11, wherein insulin resistance comprises a central metabolic change during surgical stress, hyperglycemia, decreased insulin sensitivity, stimulation of various endocrine systems, or stimulation of various inflammatory systems, or combinations thereof.

13. The method of claim 12, wherein the at least one keto ester compound is selected from 3-hydroxybutyl 3-hydroxybutanoate, 3-hydroxybutyl (3R)-3-hydroxybutanoate, 3-hydroxybutyl (R)-3-hydroxybutyrate, R-BHB; BD-AcAc 2; ester ketone, ketone ester; (R)—(R)-3-hydroxybutyl 3-hydroxybutanoate; [(3R)-3-hydroxybutyl] (3R)-3-hydroxybutanoate; (3R)-3-Hydroxybutanoic acid (3R)-3-hydroxybutyl ester; Butanoic acid, 3-hydroxy-, (3R)-3-hydroxybutyl ester, analogues and salts thereof.

14. The method of claim 13, wherein the at least one maltodextrin compound comprises a digestion-resistant maltodextrin, delayed-release maltodextrin, or slow-release maltodextrin, or combinations thereof.

15. The method of claim 14, wherein the at least one keto ester compound and the at least one maltodextrin compound are co-formulated; and wherein the co-formulated ratio of keto ester compound to maltodextrin compound is from about 1:1 to about 1:8.

16. The method of claim 15, wherein the at least one keto ester compound is 3-hydroxybutyl (3R)-3-hydroxybutanoate or 3-hydroxybutyl (R)-3-hydroxybutyrate.

17. The method of claim 16, wherein the step of providing comprises providing from at least about one hour prior to a surgical procedure.

18. A composition comprising an effective amount of at least one keto ester compound or a pharmaceutically acceptable salt thereof, and an effective amount of at least one maltodextrin compound;
wherein the at least one keto ester compound is selected from 3-hydroxybutyl 3-hydroxybutanoate, 3-hydroxybutyl (3R)-3-hydroxybutanoate, 3-hydroxybutyl (R)-3-hydroxybutyrate, R-BHB; BD-AcAc 2; ester ketone, ketone ester; (R)—(R)-3-hydroxybutyl 3-hydroxybutanoate; [(3R)-3-hydroxybutyl] (3R)-3-hydroxybutanoate; (3R)-3-Hydroxybutanoic acid (3R)-3-hydroxybutyl ester; Butanoic acid, 3-hydroxy-, (3R)-3-hydroxybutyl ester, analogues and salts thereof; and wherein the at least one maltodextrin compound is a polysaccharide comprising D-glucose units connected in chains of variable length.

19. The composition of claim 18, wherein the at least one keto ester compound and the at least one maltodextrin compound are co-formulated; and wherein the co-formulated ratio of keto ester compound to maltodextrin compound is from about 1:1 to about 1:25.

20. The composition of claim 20, wherein the at least one keto ester compound is 3-hydroxybutyl (3R)-3-hydroxybutanoate or 3-hydroxybutyl (R)-3-hydroxybutyrate.
